(19) United States
(12) Patent Application Publication
(10) Pub. No.: US 2026/0014121 A1
(43) Pub. Date: Jan. 15, 2026

(54) Compositions for Treatment of Ear Conditions

(71) Applicant: VETNIQUE LABS, LLC, Naperville, IL (US)

(72) Inventor: Jamy A. Bascharon, Naperville, IL (US)

(21) Appl. No.: 18/994,491

(22) PCT Filed: Jul. 14, 2023
(86) PCT No.: PCT/US2023/027806

§ 371 Date: Jan. 14, 2025

Related U.S. Application Data
(60) Provisional application No. 63/389,024, filed on Jul. 14, 2022.

(51) Int. Cl. A61K 31/4174; A61K 9/70; A61K 31/155; A61K 31/4166; A61K 31/573; A61K 31/60; A61K 36/484; A61K 47/36

(52) U.S. Cl. CPC A61K 31/4174; A61K 9/7007; A61K 31/155; A61K 31/4166; A61K 31/573; A61K 31/60; A61K 36/484; A61K 47/36

(57) ABSTRACT

This disclosure relates to compositions for improving ear health and treating conditions associated with a subject's ear. In particular, the disclosure relates to compositions formulated as a wipe comprising an antiseptic agent, an antifungal agent and/or a corticosteroid.

### RELATED APPLICATION

This application claims priority to U.S. Provisional Application No. 63/389,024, filed Jul. 14, 2022, the contents of which are hereby incorporated by reference in their entirety.

### FIELD OF THE DISCLOSURE

This disclosure relates to compositions for treating conditions associated with a subject's ear. In particular, the disclosure relates to compositions formulated as a wipe comprising an antiseptic agent, an antifungal agent and/or a corticosteroid. For example, the disclosure relates to compositions formulated as a wipe comprising chlorhexidine digluconate, miconazole nitrate, and/or hydrocortisone.

### BACKGROUND OF THE DISCLOSURE

Various mammals, such as dogs and cats, have long, narrow ear canals, leaving them prone to ear infections. Main causes of ear infections in such pets are allergies (causing yeast and bacteria overgrowth in the ear canals), damp ears, parasites, and waxy build-up. Routine cleaning of a pet's ears is a simple way to prevent painful and costly ear infections.

### SUMMARY OF THE DISCLOSURE

An absorbent article is provided to be topically applied to a subject's ear region. The absorbent article may take the form of a wipe, which both cleanses and dries the ear, as well as provides odor control and helps to soften ear wax. The ear and ear region is a sensitive area and can be difficult to clean without irritation to the skin; compositions described herein are able to effectively cleanse and remove excess moisture in the ear region while remaining gentle on skin.

One aspect of the disclosure provides a composition comprising an antiseptic agent in an amount between about 0.05% to about 5% by weight of the total composition and an antifungal agent in an amount between about 0.05% to about 5 wt % by weight of the total composition.

Another aspect of the disclosure provides a composition comprising chlorhexidine digluconate in an amount between about 0.05% to about 5% by weight of the total composition and miconazole nitrate in an amount between about 0.05% to about 5% by weight of the total composition.

Still another aspect of the disclosure provides a composition comprising hydrocortisone in an amount of about 0.50% by weight of the total composition, miconazole nitrate in an amount between about 0.05% to about 5% by weight of the total composition, and salicylic acid in an amount between of about 0.20% by weight of the total composition, and one or more of propylene glycol, water, SD alcohol 40B, glycerin, benzyl alcohol, PEG-75 lanolin, fragrance, allantoin, glycyrrhiza glabra (licorice root extract), propanediol, rhamnose, glucose, and glucuronic acid.

Further provided herein is a method of treatment or prevention of an ear condition, the method comprising administering to a subject in need of such treatment an effective amount of one or more compositions disclosed herein.

### DETAILED DESCRIPTION OF THE DISCLOSURE

This disclosure relates to compositions for promoting ear health and odor control. In particular, the disclosure relates to compositions formulated as an absorbent article, such as a wet wipe, comprising hydrocortisone, miconazole nitrate, and salicylic acid. The combinations disclosed herein produce synergistic ear health effects, as the wipe removes debris, such as dirt and excess wax, from the ear region, while promoting skin hydration, and all the while not irritating the sensitive ear area.

Ranges and amounts can be expressed as ‘about’ a particular value or range. About also includes the exact amount. For example, “about 5%” means “about 5%” and also “5%.” The term “about” can also refer to +10% of a given value or range of values. Therefore, about 5% also means 4.5%- 5.5%, for example.

The term “treating” or “treatment” is used herein, for instance, in reference to methods of cleansing an ear or region around the ear, or treating an ear condition, and generally includes the administration of a compound or composition which reduces the frequency of, or prevents the onset of, symptoms of a medical condition or enhances the condition or hydration of the intended treatment area in a subject relative to the subject not receiving the compound or composition. Treating or treatment can include routine application for maintenance of a cleansed and healthy ear region.

The term “subject” is used herein to refer to a mammalian subject.

Chlorhexidine digluconate (CAS RN: 18472-51-0) is the gluconate salt form of chlorhexidine, a biguanide compound used as an antiseptic agent with topical antibacterial activity. In some embodiments, the disclosure provides a composition, wherein chlorhexidine digluconate is in the amount of about 0.05% to about 5% weight of the composition (wt %). In some embodiments, the disclosure provides a composition, wherein chlorhexidine digluconate is in the amount of about 0.1 to about 3 wt %. In other embodiments, the chlorhexidine digluconate is in the amount of about 0.1 to about 1 wt %. In other embodiments, the chlorhexidine digluconate is in the amount of about 0.1 wt %. In other embodiments, the chlorhexidine digluconate is in the amount of about 0.2 wt %.

Miconazole nitrate (CAS RN: 22832-87-7) belongs to the group of medicines called antifungals. In some embodiments, the disclosure provides a composition, wherein miconazole nitrate is in the amount of about 0.05 to about 5 wt %. In some embodiments, the disclosure provides a composition, wherein miconazole nitrate is in the amount of about 0.1 to about 5 wt %. In other embodiments, the miconazole nitrate is in the amount of about 0.2 to about 1 wt %. In other embodiments, the miconazole nitrate is in the amount of about 0.2 wt %.

In some embodiments, the disclosure provides a composition comprising chlorhexidine digluconate in the amount between about 0.05 to about 5 wt % and miconazole nitrate in the amount between about 0.05 to about 5 wt %. In some embodiments, the chlorhexidine digluconate is in the amount of about 0.2 wt % and miconazole nitrate is in the amount of about 0.2 wt %.

Hydrocortisone (CAS RN: 50-23-7) is a steroid (corticosteroid) medicine. In some embodiments, the disclosure provides a composition, wherein hydrocortisone is in the amount of about 0.1 to about 5 wt %. In other embodiments, hydrocortisone is in the amount of about 0.1 to about 3 wt %. In other embodiments, hydrocortisone in the amount of about 0.1 to about 1 wt %. In other embodiments, hydrocortisone is in the amount of about 0.1 wt %. In other embodiments, hydrocortisone is in the amount of about 0.2 wt %. In other embodiments, hydrocortisone is in the amount of about 0.5 wt %.

Salicylic acid (CAS RN: 69-72-7) is a monohydroxybenzoic acid that serves as an antiinfective agent and an antifungal agent. In some embodiments, the disclosure provides a composition, wherein salicylic acid is in the amount of about 0.05 to about 5 wt %. In some embodiments, the disclosure provides a composition, wherein salicylic acid is in the amount of about 0.1 to about 5 wt %. In other embodiments, the salicylic acid is in the amount of about 0.1 to about 1 wt %. In other embodiments, the salicylic acid is in the amount of about 0.2 wt %.

In some embodiments, the disclosure provides a composition comprising chlorhexidine digluconate in the amount between about 0.05 to about 5 wt %, miconazole nitrate in the amount between about 0.05 to about 5 wt %, and hydrocortisone in the amount between about 0.1 to about 5 wt %. In some embodiments, the disclosure provides a composition wherein chlorhexidine digluconate is in the amount of about 0.2 wt %, miconazole nitrate in is the amount of about 0.2 wt %, and hydrocortisone in is the amount of about 0.1 wt %.

In some embodiments, the disclosure provides a composition comprising chlorhexidine digluconate in the amount between about 0.05 to about 5 wt %, miconazole nitrate in the amount between about 0.05 to about 5 wt %, and hydrocortisone in the amount between about 0.1 to about 5 wt %. In some embodiments, the disclosure provides a composition wherein chlorhexidine digluconate is in the amount of about 0.2 wt %, miconazole nitrate in is the amount of about 0.2 wt %, and hydrocortisone in is the amount of about 0.2 wt %.

In some embodiments, the disclosure provides a composition comprising chlorhexidine digluconate in the amount between about 0.05 to about 5 wt %, miconazole nitrate in the amount between about 0.05 to about 5 wt %, and hydrocortisone in the amount between about 0.1 to about 5 wt %. In some embodiments, the disclosure provides a composition wherein chlorhexidine digluconate is in the amount of about 0.2 wt %, miconazole nitrate in is the amount of about 0.2 wt %, and hydrocortisone in is the amount of about 0.5 wt %.

In some embodiments, the disclosure provides a composition comprising salicylic acid in the amount between about 0.05 to about 5 wt %, miconazole nitrate in the amount between about 0.05 to about 5 wt %, and hydrocortisone in the amount between about 0.1 to about 5 wt %. In some embodiments, the disclosure provides a composition wherein salicylic acid is in the amount of about 0.2 wt %, miconazole nitrate in is the amount of about 0.2 wt %, and hydrocortisone in is the amount of about 0.1 wt %.

In some embodiments, the disclosure provides a composition comprising salicylic acid in the amount between about 0.05 to about 5 wt %, miconazole nitrate in the amount between about 0.05 to about 5 wt %, and hydrocortisone in the amount between about 0.1 to about 5 wt %. In some embodiments, the disclosure provides a composition wherein salicylic acid is in the amount of about 0.2 wt %, miconazole nitrate in is the amount of about 0.2 wt %, and hydrocortisone in is the amount of about 0.2 wt %.

In some embodiments, the disclosure provides a composition comprising salicylic acid in the amount between about 0.05 to about 5 wt %, miconazole nitrate in the amount between about 0.05 to about 5 wt %, and hydrocortisone in the amount between about 0.1 to about 5 wt %. In some embodiments, the disclosure provides a composition wherein salicylic acid is in the amount of about 0.2 wt %, miconazole nitrate in is the amount of about 0.2 wt %, and hydrocortisone in is the amount of about 0.5 wt %.

In some embodiments, the composition further comprises one or more of allantoin, licorice root extract, and a polysaccharide. In some embodiments, the polysaccharide is a natural branched polysaccharide, optionally a rhamnose-rich polysaccharide, obtained by bacterial fermentation. In some embodiments, the polysaccharide is TEFLOSER® (available from Solabia Group, Pantin, France)

Allantoin (CAS RN: 97-59-6) promotes and accelerates cell proliferation and provides keratolytic and soothing action and has a beneficial influence on skin moisture. In some embodiments, the disclosure provides a composition, wherein allantoin is in the amount of about 0.01 to about 5 wt %. In other embodiments, allantoin is in the amount of about 0.1 to about 5 wt %. In another embodiment, allantoin is in the amount of about 0.1 to about 1 wt %. In other embodiments, allantoin is in the amount of about 0.15 wt %.

Glycyrrhiza glabra (licorice) root extract is the polysaccharide fraction of licorice and has immunomodulatory activity by strengthening phagocytosis in the endothelial reticular system and stimulating interferon production. In some embodiments, the disclosure provides a composition, wherein the licorice root extract is in the amount of about 0.05 to about 5 wt %. In some embodiments, the disclosure provides a composition, wherein the licorice root extract is in the amount of about 0.05 to about 1 wt %. In some embodiments, the disclosure provides a composition, wherein the licorice root extract is in the amount of about 0.1 to about 1 wt %. In other embodiments, the licorice root extract is in the amount of about 0.25 to about 1 wt %. In other embodiments, the licorice root extract is in the amount of about 0.25 wt %.

Polysaccharides such as rhamnose, glucose, glucuronic acid, alpha-glucan oligosaccharide protect microbiota equilibrium, inhibit biofilm formation and prevents adhesion of undesirable and/or pathogenic bacteria on skin's surface. In some embodiments, the polysaccharide is in the amount of about 0.01 to about 5 wt %. In other embodiments, the polysaccharide is in the amount of about 0.01 to about 1 wt %. In other embodiments, the polysaccharide is in the amount of about 0.05 to about 1 wt %. In other embodiments, the polysaccharide is in the amount of about 0.05 wt %.

In some embodiments, the disclosure provides a composition, wherein allantoin is in the amount between about 0.01 to about 5 wt %, the licorice root extract is the amount between about 0.05 to about 5 wt %, and polysaccharide is in the amount between about 0.01 to about 5 wt %.

In some embodiments, the disclosure provides a composition wherein chlorhexidine digluconate is in the amount of about 0.2 wt %, miconazole nitrate is in the amount of about 0.2 wt %, allantoin is in the amount of about 0.15 wt %, licorice root extract is in the amount of about 0.25 wt %, and the polysaccharide is in the amount of about 0.05 wt %.

In some embodiments, the disclosure provides a composition wherein the chlorhexidine digluconate is in the amount of about 0.2 wt %, miconazole nitrate is in the amount of about 0.2 wt %, hydrocortisone is in the amount of about 0.5 wt %, allantoin is in the amount of about 0.15 wt %, licorice root extract is in the amount of about 0.25 wt %, and the polysaccharide is in the amount of about 0.05 wt %.

In some embodiments, the disclosure provides a composition wherein salicylic acid is in the amount of about 0.2 wt %, miconazole nitrate is in the amount of about 0.2 wt %, allantoin is in the amount of about 0.15 wt %, licorice root extract is in the amount of about 0.25 wt %, and the polysaccharide is in the amount of about 0.05 wt %.

In some embodiments, the disclosure provides a composition wherein the salicylic acid is in the amount of about 0.2 wt %, miconazole nitrate is in the amount of about 0.2 wt %, hydrocortisone is in the amount of about 0.5 wt %, allantoin is in the amount of about 0.15 wt %, licorice root extract is in the amount of about 0.25 wt %, and the polysaccharide is in the amount of about 0.05 wt %.

In some embodiments, the disclosure provides a composition wherein the salicylic acid is in the amount of about 0.2 wt %, miconazole nitrate is in the amount of about 0.2 wt %, hydrocortisone is in the amount of about 0.1 wt %, allantoin is in the amount of about 0.15 wt %, licorice root extract is in the amount of about 0.25 wt %, and the polysaccharide is in the amount of about 0.05 wt %.

In other embodiments, the composition may comprise additional ingredients as appropriate that would be well known to one of ordinary skill in the art such as penetration enhancers, humectants, lubricants, color, fragrance, preservatives, antioxidants, chelators, neutralizers, astringents, binders, catalysts, stabilizers, emollients, emulsifiers, surfactants, essential oils, plant/botanical extracts, conditioners, film formers, gelling agents, foaming agents, exfoliants, vitamins, minerals, pH adjusters, proteins, peptides, tactile enhancers, saccharides, solvents or any combination thereof.

In some embodiments, the composition disclosed herein may be formulated as a wipe, spray, cream, lotion, gel, fluid/liquid, soap, or any other form known in the art. In some embodiments, the composition is formulated as a wipe.

In some embodiments, the disclosure provides methods of treating or preventing ear conditions, the method comprising administering to a subject in need of such treatment an effective amount of one or more of compositions of the disclosure. In some embodiments, the ear condition is an ear infection, including bacterial, fungal and parasitic infections. Any disclosed composition, or a combination of compositions may be used in these methods.

The disclosure provides methods as described above, wherein the subject is a mammalian subject. In one embodiment, the subject is a pet. In one embodiment, the subject is a dog or a cat. In another embodiment, the subject is a dog. In other embodiment, the subject is a cat.

In some embodiments, in the methods as described above, the composition is administered over the course of a period of time. In some embodiments, the composition may be applied on a daily basis, either once a day or several times per day, over the course of days, weeks, months or years. In some embodiments, in the methods as described above, the administration is episodically, for example on an as needed basis when the subject is experiencing ear problems for a short-term period of several months.

The compositions described herein will generally be used in an amount effective to achieve the intended result, for example in an amount effective to treat or prevent the ear condition. By therapeutic benefit it is meant eradication or amelioration of the ear condition and/or eradication or amelioration of one or more of the symptoms associated with the ear condition such that the subject shows an improvement in feeling or condition, notwithstanding that the subject may still be afflicted with the condition. Therapeutic benefit also generally includes halting or slowing the progression of the ear condition, regardless of whether improvement is realized.

### EXAMPLES

The compositions and methods of the disclosure are further illustrated by the following examples, which are not to be construed as limiting the disclosure in scope or spirit to the specific procedures and compositions described in them.

A composition is formulated into a wipe by mixing the ingredients at room temperature to obtain a solution, followed by soaking an absorbent wipe substrate in the solution until the solution is absorbed onto the wipe. Representative specific, non-limiting compositions that are present per wipe are listed in Table 1, Table 2, Table 3, and Table 4.

TABLE 1
3-in-1 Medicated Wipe

| Actives | Amt (mg/wipe) | wt % |
|---|---|---|
| Chlorhexidine Digluconate | | 0.20% |
| Miconazole Nitrate | | 0.20% |
| Hydrocortisone | | 0.50% |
| Allantoin | 0.000061 | 0.15% |
| Glycyrrhiza Glabra (Licorice) Root Extract | 0.000305 | 0.25% |
| Polysaccharides (Teflose) | 0.000183 | 0.05% |

TABLE 2
3-in-1 Medicated Wipe

| Actives | Amt (mg/wipe) | wt % |
|---|---|---|
| Chlorhexidine Digluconate | | 0.20% |
| Miconazole Nitrate | | 0.20% |
| Allantoin | 0.000061 | 0.15% |
| Glycyrrhiza Glabra (Licorice) Root Extract | 0.000305 | 0.25% |
| Polysaccharides (Teflose) | 0.000183 | 0.05% |

TABLE 3
3-in-1 Medicated Wipe

| | | |
|---|---|---|
| Actives | Amt (mg/wipe) | wt % |

| | |
|---|---|
| Hydrocortisone | 0.50% |
| Miconazole Nitrate | 0.20% |
| Salicylic Acid | 0.20% |

TABLE 4
3-in-1 Medicated Wipe

| | | |
|---|---|---|
| Actives | Amt (mg/wipe) | wt % |

| | |
|---|---|
| Hydrocortisone | 0.10% |
| Miconazole Nitrate | 0.20% |
| Salicylic Acid | 0.20% |

In the composition of Tables 3 and 4, additional ingredients comprising one or more of propylene glycol, water, SD alcohol 40B, glycerin, benzyl alcohol, PEG-75 lanolin, fragrance, allantoin, glycyrrhiza glabra (licorice root extract), propanediol, rhamnose, glucose, and glucuronic acid may be present.

It is understood that the examples and embodiments described herein are for illustrative purposes only. Unless clearly excluded by the context, all embodiments disclosed for one aspect of the invention can be combined with embodiments disclosed for other aspects of the invention, in any suitable combination. It will be apparent to those skilled in the art that various modifications and variations can be made to the present invention without departing from the scope of the invention. Thus, it is intended that the present invention cover the modifications and variations of this invention provided they come within the scope of the appended claims and their equivalents.

1. A composition comprising salicylic acid in an amount between about 0.05% to about 5% by weight of the total composition and miconazole nitrate in an amount between about 0.05% to about 5 wt % by weight of the total composition.

2. The composition of claim 1 further comprising hydrocortisone in an amount between about 0.1 to about 5 wt % by weight of the total composition.

3. The composition of claim 2, wherein salicylic acid is in the amount of about 0.2% by weight of the total composition, miconazole nitrate is in the amount of about 0.2% by weight of the total composition, and hydrocortisone in is the amount of about 0.1% by weight of the total composition.

4. The composition of claim 1 further comprising one or more of allantoin, licorice root extract, and polysaccharide.

5. The composition of claim 4, wherein allantoin is an amount between about 0.0 1% to about 5% by weight of the total composition, the licorice root extract is an amount between about 0.05% to about 5% by weight of the total composition, and the polysaccharide is an amount between about 0.01% to about 5% by weight of the total composition.

6. The composition of claim 4, wherein salicylic acid is in the amount of about 0.2% by weight of the total composition, miconazole nitrate is in the amount of about 0.2% by weight of the total composition, hydrocortisone is in the amount of about 0.5% by weight of the total composition, allantoin is in the amount of about 0.15% by weight of the total composition, the licorice root extract is in the amount of about 0.25% by weight of the total composition, and the polysaccharide is in the amount of about 0.05% by weight of the total composition.

7. The composition of claim 1, wherein the composition is formulated as a wipe.

8. A method of treatment or prevention of an ear condition, the method comprising administering to a subject in need of such treatment an effective amount of one or more compositions of claim 1.

9. The method of claim 8, wherein the subject is a dog or a cat.

10. The method of claim 8, wherein the ear condition is an infection.

11. A composition comprising chlorhexidine digluconate in an amount between about 0.05% to about 5% by weight of the total composition and miconazole nitrate in an amount between about 0.05% to about 5 wt % by weight of the total composition.

12. The composition of claim 11 further comprising hydrocortisone in an amount between about 0.1 to about 5 wt % by weight of the total composition.

13. The composition of claim 12, wherein chlorhexidine digluconate is in the amount of about 0.2% by weight of the total composition, miconazole nitrate is in the amount of about 0.2% by weight of the total composition, and hydrocortisone in is the amount of about 0.1% by weight of the total composition.

14. The composition of claim 11 further comprising one or more of allantoin, licorice root extract, and polysaccharide.

15. The composition of claim 14, wherein allantoin is an amount between about 0.0 1% to about 5% by weight of the total composition, the licorice root extract is an amount between about 0.05% to about 5% by weight of the total composition, and the polysaccharide is an amount between about 0.01% to about 5% by weight of the total composition.

16. The composition of claim, wherein chlorhexidine digluconate is in the amount of about 0.2% by weight of the total composition, miconazole nitrate is in the amount of about 0.2% by weight of the total composition, hydrocortisone is in the amount of about 0.1% by weight of the total composition, allantoin is in the amount of about 0.15% by weight of the total composition, the licorice root extract is in the amount of about 0.25% by weight of the total composition, and the polysaccharide is in the amount of about 0.05% by weight of the total composition.
